(19) United States
(12) Patent Application Publication
(10) Pub. No.: US 2019/0327584 A1
(43) Pub. Date: Oct. 24, 2019

(54) Home Dialysis Management Using a Connected Health System Network

(71) Applicant: Fresenius Medical Care Holdings, Inc., Waltham, MA (US)

(72) Inventors: Kulwinder S. Plahey, Martinez, CA (US); Teresa J. Close, Willis, TX (US); John A. Biewer, Waltham, MA (US)

(21) Appl. No.: 15/956,422

(22) Filed: Apr. 18, 2018

(51) Int. Cl. H04W 4/12; H04L 12/58; A61M 1/14

(52) U.S. Cl. CPC H04W 4/12; H04W 88/16; A61M 1/14; H04L 51/10

(57) ABSTRACT

Medical devices have the ability to connect through a secure gateway to a network, including both local and external networks. Transmissions over these networks may be used to inform, remind and/or alert a patient about tasks related to a medical treatment performed on a medical device, such as a peritoneal dialysis machine, at the home of the patient.

### TECHNICAL FIELD

This application relates generally to systems and methods for medical treatment and device management using transmissions over a network system.

### BACKGROUND

Medical devices such as dialysis machines are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate, or dialysis solution. The membranous lining of the patient's peritoneum acts as a natural semi-permeable membrane that allows diffusion and osmosis exchanges to take place between the solution and the blood stream. Automated peritoneal dialysis machines, also called PD cyclers, are designed to control the entire peritoneal dialysis process so that it can be performed at home, usually overnight, without clinical staff in attendance. Both HD and PD machines may include displays with touch screens or other user interfaces that display information of a dialysis treatment and/or enable an operator or patient to interact with the machine.

Patients may have many treatment steps during dialysis therapy and may have different steps required on different days, which may cause confusion, mistakes or omitted steps. For example, patients may be able to disconnect from a PD cycler for a period of time during treatment and/or may have manual PD exchanges to perform away from a cycler on certain days. During these times away from the cycler, there may be prescribed steps and times that a patient must remember. When away from the machine, a patient may become distracted and not perform the required steps timely or effectively.

Accordingly, it would be desirable to provide a system that addresses the above-noted concerns and other issues.

### SUMMARY

According to an embodiment of the system described herein, a medical system includes a medical device having a first display and a processing device remotely located from the medical device. A network messaging system transmits a message over a network between the medical device and the remote processing device, and in which the message includes information concerning a medical treatment performed by the medical device.

In one or more implementations, the medical device is a dialysis machine that is located in the home, and the medical treatment is a dialysis treatment. A gateway device may be provided that facilitates a wireless network connection with the medical device. The gateway, the medical device and the processing device may be joined to a same wireless local area network. The processing device may be a mobile device and/or a server. The message may include an instruction or a reminder to a patient concerning the medical treatment, may include one or more of an audio or video message, and/or the message may be interactive and require at least one input from the patient. The message may be provided over the Internet via a network infrastructure.

According to another embodiment of the system described herein, a network messaging system for a medical device includes a message-generating component that transmits a message over a network relating to a medical treatment performed by the medical device and a message-receiving component that receives the message. A gateway device is provided that facilitates a wireless connection to the medical device, and in which the message is transmitted by the gateway device to or from the medical device.

In one or more implementations, the medical device may be a dialysis machine and the medical treatment may be a dialysis treatment. The gateway and the medical device may be located in a home of a patient. The message-generating component may be a component of the medical device and/or the message-receiving component may be a component of the medical device. In other implementations, the message-receiving component may be a component of a mobile device. The message may include an instruction or a reminder to a patient concerning the medical treatment and/or may include one or more an audio message, a video message or an interactive message that requires at least one input from the patient. The message may be provided over the Internet via a network infrastructure.

### BRIEF DESCRIPTION OF THE DRAWINGS

Embodiments and features of the system described herein are explained with reference to the several figures of the drawings, which are briefly described as follows.

FIG. 1 illustrates an exemplary embodiment of a dialysis machine in a dialysis system configured in accordance with the present disclosure.

FIG. 2 illustrates another exemplary embodiment of a dialysis machine in accordance with the present disclosure.

FIG. 3 is a schematic illustration showing an example of a connected health (CH) system that may include, among other things, a processing system, a CH cloud service and a CH gateway that may be used in connection with the system described herein.

FIG. 4 is a schematic illustration showing one or more implementations of a network messaging system implemented with a medical system according to an embodiment of the system described herein.

FIG. 5 is a schematic illustration showing another example of a network messaging system according to an embodiment of the system described herein.

### DETAILED DESCRIPTION

According to the system described herein, medical devices have the ability to connect through a secure gateway to a network, including both local and external networks. Transmissions over these networks may be used to inform, remind and/or alert a patient about tasks related to a medical treatment.

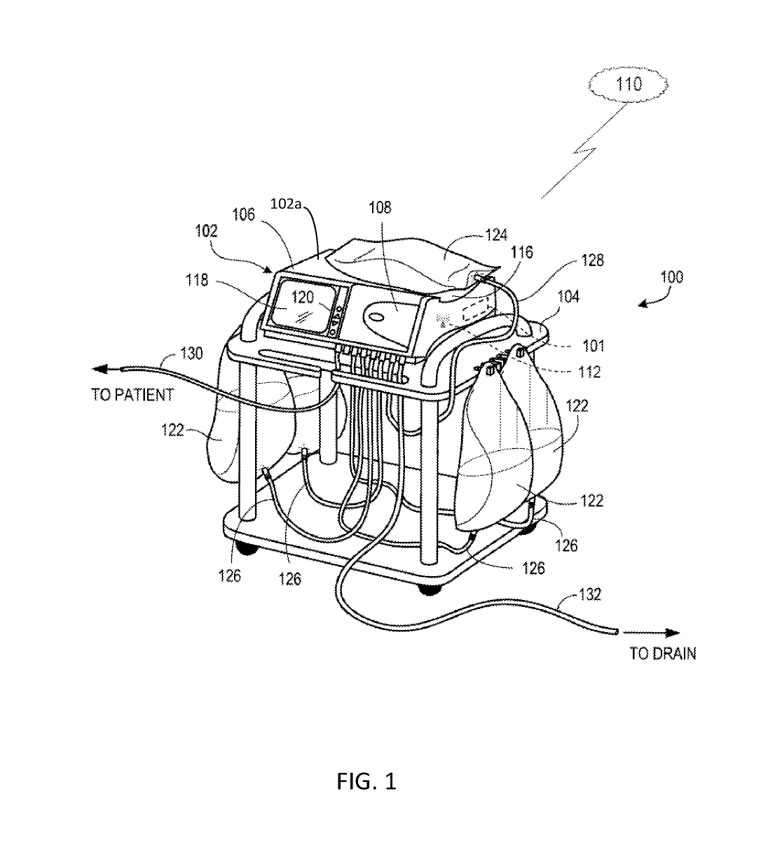

FIG. 1 shows an example of a medical device, implemented as a peritoneal dialysis (PD) system 100, that is configured in accordance with an exemplary embodiment of the system described herein. In some implementations, the PD system 100 may be configured for use at a patient's home (e.g., a home dialysis system). The PD system 100 may include a dialysis machine 102 (e.g. a PD machine, also referred to as a PD cycler) and in some embodiments may be seated on a cart 104. The dialysis machine 102 may include a housing 106, a door 108, and a cartridge interface for contacting a disposable PD cassette, or cartridge, when the cartridge is disposed within a compartment formed between the cartridge interface and the closed door 108. A heater tray 116 may be positioned on top of the housing 106. The heater tray 116 may be any size and shape to accommodate a bag of dialysate (e.g., a 5L bag of dialysate). The dialysis machine 102 may also include a user interface such as a touch screen 118 and control panel 120 operable by a user (e.g., a caregiver or a patient) to allow, for example, set up, initiation, and/or termination of a PD treatment.

Dialysate bags 122 may be suspended from the sides of the cart 104, and a heater bag 124 may be positioned in the heater tray 116. Hanging the dialysate bags 122 may improve air management as any air is disposed by gravity to a top portion of the dialysate bag 122. Valves may be attached to a bottom portion of the dialysate bags 122 so fluid is drawn out and air delivery is minimized. Dialysate from the dialysate bags 122 may be transferred to the heater bag 124 in batches. For example, a batch of dialysate may be transferred from the dialysate bags 122 to the heater bag 124, where the dialysate is heated by the heating element. When the batch of dialysate has reached a predetermined temperature (e.g., approximately 98°-100° F., 37° C.), the batch of dialysate may be flowed into the patient. The dialysate bags 122 and the heater bag 124 may be connected to the cartridge via dialysate bag lines 126 and a heater bag line 128, respectively. The dialysate bag lines 126 may be used to pass dialysate from dialysate bags 122 to the cartridge during use, and the heater bag line 128 may be used to pass dialysate back and forth between the cartridge and the heater bag 124 during use. In addition, a patient line 130 and a drain line 132 may be connected to the cartridge. The patient line 130 may be connected to a patient's abdomen via a catheter and may be used to pass dialysate back and forth between the cartridge and the patient's peritoneal cavity during use. The drain line 132 may be connected to a drain or drain receptacle and may be used to pass dialysate from the cartridge to the drain or drain receptacle during use.

The touch screen 118 and the control panel 120 may allow a user to input various treatment parameters to the dialysis machine 102 and to otherwise control the dialysis machine 102. In addition, the touch screen 118 may serve as a display. The touch screen 118 may function to provide information to the patient and the operator of the PD system 100. For example, the touch screen 118 may display information related to a dialysis treatment to be applied to the patient, including information related to a prescription. In various embodiments, the control panel 120 may also include audio and video component capabilities, including speakers, microphones and/or cameras.

The dialysis machine 102 may include a processing module 101 that resides inside the dialysis machine 102, the processing module 101 being configured to communicate with the touch screen 118 and the control panel 120. The processing module 101 may be configured to receive data from the touch screen 118 the control panel 120 and sensors, e.g., temperature and pressure sensors, and control the dialysis machine 102 based on the received data. For example, the processing module 101 may adjust the operating parameters of the dialysis machine 102.

The dialysis machine 102 may be configured to connect to a network 110. The connection to network 110 may be via a wireless connection, or in some cases a wired connection, as further discussed elsewhere herein. The dialysis machine 102 may include a connection component 112 configured to facilitate the connection to the network 110. The connection component 112 may be a transceiver for wireless connections and/or other signal processor for processing signals transmitted and received over a wired connection. In the case of a wired connection, the connection component 112 may be a port enabling a physical connection to a network component. Other medical devices (e.g., other dialysis machines) or components may be configured to connect to the network 110 and communicate with the dialysis machine 102. Although discussed herein principally in connection with a peritoneal dialysis machine, the system described herein may be used and implemented in connection with other types of medical devices having one or more displays, including home hemodialysis machines and/or other home medical devices.

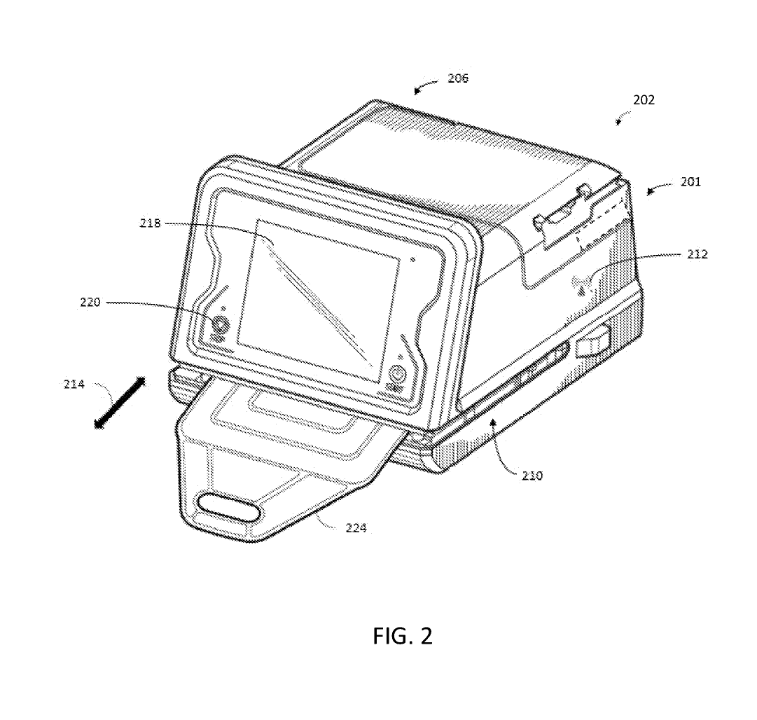

FIG. 2 is a schematic illustration showing another exemplary embodiment of a dialysis machine 202 in accordance with the present disclosure. The dialysis machine 202 may be implemented in the peritoneal dialysis system 100 and may have at least some similar components as that of the dialysis machine 102, for example, including a housing 206, a processing module 201, a connection component 212, a touch screen 218, and a control panel 220 operable by a user (e.g., a caregiver or a patient) to allow, for example, set up, initiation, and/or termination of a PD treatment. The processing module 201 and the connection component 212 may be configured similarly to the processing module 101 and connection component 112 described above. However, instead of a heater tray being positioned on a top surface 102a of the housing as shown in FIG. 1 for the dialysis machine 102, one or more heating elements may be disposed internal to the machine 202. For example, a warmer pouch 224 may be insertable into an opening 210 in a direction indicated at arrow 214. In embodiments, the warmer pouch 224 may be configured so dialysate may continually flow through the warmer pouch (instead of transferred in batches) to achieve a predetermined temperature before flowing into the patient.

Medical devices may have the ability to connect through a secure gateway to connect to a network, including a network outside the home to send and receive information to a clinic. The connection, network and data transmissions among components, both local and external, may be controlled and/other otherwise incorporated into a system that facilitates such functions with appropriate network infrastructure, and which may, in some implementations, be referred to as a connected health system. For further descriptions of systems for securely connecting, pairing and/or monitoring medical devices, such as in a connected health system, reference is made to US Pub. No. 20160206800 entitled “Remote Monitoring Interface Device and Mobile Application for Medical Devices” to Tanenbaum et al., U.S. Pat. No. 9,800,663 entitled “Associating Dialysis Accessories Using Near Field Communication” to Arrizza, US Pub. No. 20170087290 entitled “Short-Range Wireless Communication for a Dialysis System” to Medina et al., US Pub. No. 20170076069 entitled “Secure Network-Based System for Communication of Clinical Data” to Moissl et al., and U.S. Pat. No. 9,178,891 entitled “Remote Control of Dialysis Machines” to Wang et al., the disclosures of all of which are hereby incorporated by reference.

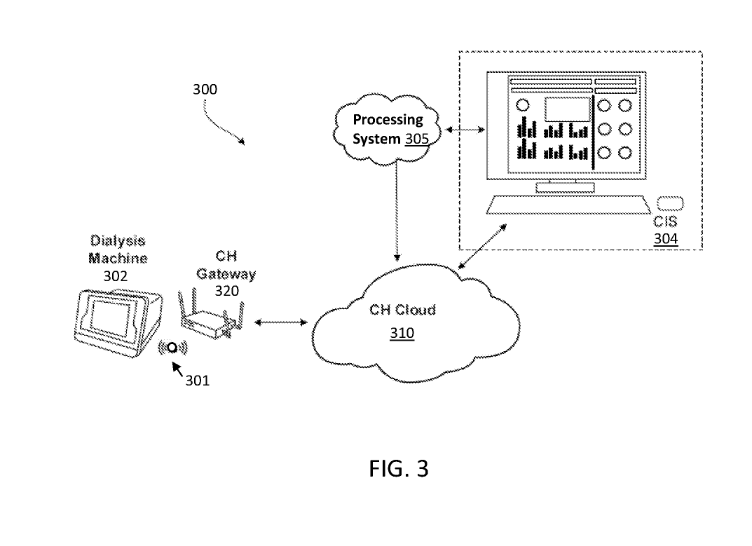

FIG. 3 is a schematic illustration showing an example of a connected health (CH) system 300 that may include, among other things, a processing system 305, a CH cloud service 310 and a gateway (CH Gateway) 320 that may be used in connection with network aspects of the system described herein. The processing system 305 may be a server and/or cloud-based system that processes, compatibility checks and/or formats medical information, including prescription information generated at a clinical information system (CIS) 304 of a clinic or hospital, in connection with data transmission operations of the CH system 300. The CH system 300 may include appropriate encryption and data security mechanisms. The CH cloud service 310 may be a cloud-based application that serves as a communication pipeline (e.g., facilitates the transfer of data) among components of the CH system 300 via connections to a network such as the Internet. The gateway 320 may serve as a communication device facilitating communication among components of the CH system 300. In various embodiments, the gateway 320 is in communication with the dialysis machine 302 via a wireless connection 301, such as a Bluetooth, Wi-Fi and/or other appropriate type of local or short range wireless connection. The dialysis machine 302 may be similar to one or more of the dialysis machines 102, 202 discussed elsewhere herein. The gateway 320 may also be in connection with the CH cloud service 310 via a secure network (e.g. Internet) connection. The gateway 320 is configured to transmit/receive data to/from the CH cloud service 310 and transmit/receive data to/from the dialysis machine 302. The dialysis machine 302 may poll the CH cloud service 310 for available files (e.g., via the gateway 320), and the dialysis machine 302 may temporarily store available files for processing.

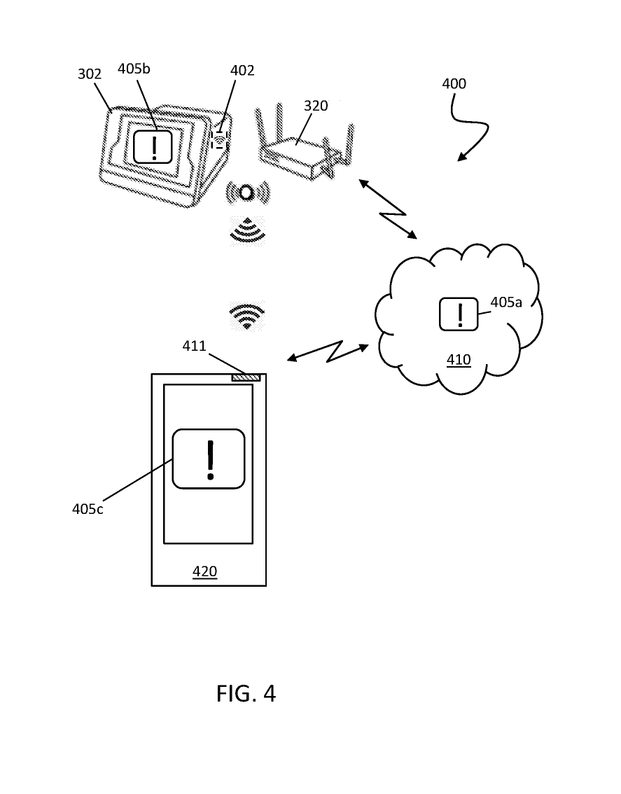

FIG. 4 is a schematic illustration showing one or more implementations of a network messaging system 400 according to an embodiment of the system described herein. A network 410 is illustrated schematically representing an intranet or external network, such as the Internet, through which messages, such as a message 405a that may be a reminder, alert and/or other message concerning a medical treatment, may be transmitted from one or more entities and/or software systems to or via one or more components of a medical system including the dialysis machine 302. The message 405a may be transmitted using one or more components of the CH system 300, such as via the gateway 320, to the dialysis machine 302 and including wireless transmission via a wireless transceiver 412 of the dialysis machine 302. The message is shown displayed as a message 405b on the dialysis machine 302 and which may be the same as the message 405a or a different, expanded or condensed version of the message 405a. In an embodiment, the message 405b displayed on the dialysis machine 302 may be an alert or reminder to a patient or caregiver concerning an aspect of a dialysis treatment or therapy to be performed using the dialysis machine 302. In various embodiments, one or more of the messages 405a,b,c may include photographic, audio and/or video messages, and may include dynamic or interactive elements using graphical user interface (GUI) components (e.g. touchscreen) of the displaying screen, that, for example, require the patient to select options to proceed through the interactive elements of the message.

In another embodiment, also illustrated by the network messaging system 400, the network message 405a may be transmitted to a portable or mobile device 420, via a wireless transceiver 411 of the mobile device 420, and which may display a message 405c that may be the same as the message 405a and/or the message 405b or a different, expanded or condensed version of the message 405a and/or the message 405b. In various embodiments, the mobile device 420 may include a smartphone, a tablet device and/or other mobile computing device having a display.

In another embodiment, the network message 405b displayed on the dialysis machine 302 may be transmitted from the dialysis machine 302 to the mobile device 420 either directly or via the gateway 320. For example, the network message 405b may be a reminder concerning an aspect of the dialysis treatment that is generated at the dialysis machine 302 and displayed on the dialysis machine 302 and transmitted to the mobile device 420 of the patient. Additionally, and/or alternatively, in another embodiment, the gateway 320 may generate the network message and transmit the message to the dialysis machine 302, where the message is displayed as the message 405b, and/or the gateway 320 may transmit the message to the mobile device 420, where the message is displayed at the message 405b. In yet another embodiment, the gateway 320 and/or the dialysis machine 302 may generate the network message and transmit the message via the connected health system 300 and/or other wide area network to a remote location, such as to the CIS 304 where the message may be displayed as the display message 405a.

In one or more embodiments, the message 405a may be transmitted via a cloud-based or wide area network communication system to the gateway 320 and thereafter transmitted via a local area network, such as a Wi-Fi or Bluetooth connection, to the dialysis machine 302 or mobile device 420. Additionally, and/or alternatively, in another embodiment, the message 405a may be transmitted via a mobile telecommunication system to the mobile device 420 and thereafter transmitted via a local area network, such as a Wi-Fi or Bluetooth connection, to dialysis machine 302 either directly or via the gateway 320. In an embodiment, the mobile device 420 may include and run a mobile application that enables communication between the mobile device 420 and the gateway 320 and/or dialysis machine 302. The communication may be suitably encrypted/decrypted and the processing may include authorized pairing between the mobile application/the mobile device 420 and the gateway 320 and/or the dialysis machine 302. References or descriptions of systems for securely connecting, pairing and/or monitoring medical devices, such as in a connected health system, are further described elsewhere herein.

According to implementations and embodiments of the system described herein, the wireless connection of the gateway 320 may be used to send, transmit and/or otherwise convey the message 405a that may be an Internet message, a short message service (SMS) message, a multimedia messaging service (MMS) message, an email and/or other messaging path to inform, alert or remind the patient about a time sensitive task related to a treatment. For example, the message may advise or otherwise provide information about: returning from a PD disconnect at a certain time; setting up device/gathering supplies/preparing for a treatment; a timer or alarm clock; sending a message on treatment completion to patient, caregiver, clinic etc.; a manual exchange reminder; a reminder about an injectable into a solution bag (e.g. antibiotics); a reminder to take effluent samples; a reminder to connect a drain bag vs a drain line; a reminder not to drain solutions into drain bag at end of treatment, and/or other appropriate messages or reminders concerning a dialysis treatment.

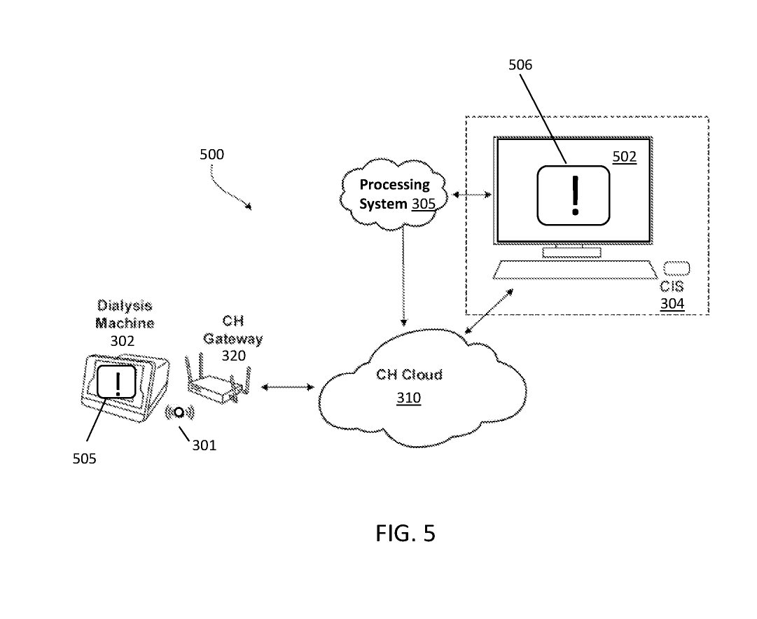

FIG. 5 is a schematic illustration showing another implementation of a network messaging system 500 according to an embodiment of the system described herein. The network messaging system 500 may include use of components of the connected health system 300 discussed elsewhere herein, including the gateway 320, CH cloud 310 and/or processing system 305. The dialysis machine 302 may include a displayed message 505 that has been received according to one or more of the embodiments discussed elsewhere herein. For example, the displayed message 505 may be a version of a message 506 that has been input via a computer 502 at the clinical information system (CIS) 304 of a remotely located clinic. For example, the message 506 may be an email message that has been generated at the CIS 304.

Additionally, and/or alternatively, in another embodiment, the displayed message 505 may be information generated at the dialysis machine 302, information responsive to previously received reminder and/or a confirmation of an action taken in response to a reminder. The information of the displayed message 505 may then be transmitted from the dialysis machine 302, via the one or more components of the CH system 300, and received and displayed as the message 506 on the computer 502 of the CIS 304.

The following are specific examples of network messaging that may be implemented according to the system described herein.

### Example 1: Medication Reminder when Patient Away from Dialysis Machine

The network messaging aspects of the system described herein may be used to transmit a message about a time sensitive medication task for when the patient is away from the dialysis machine 302. In an embodiment, the dialysis machine 302 may have stored thereon (e.g. from a prior prescription download) the full prescription for the required medication to be taken by the patient and may transmit the information of the medication reminder to the mobile device 420 of the patient either directly or via the gateway 320. Additionally, and/or alternatively, the gateway 320 may have stored thereon (e.g. from a prior prescription download) the full prescription and can send the medication reminder message either directly to the mobile device 420 of the patient, to the dialysis machine 302 for visual display thereon, or to both.

### Example 2: Medication Reminder for Injection into Dialysate Bags

The network messaging aspects of the system described herein may be used to transmit a message that a patient needs to add an injectable, such as an antibiotic, into a dialysate bag. In an embodiment, the dialysis machine 302 may receive the reminder for the required medication to be injected by the patient via the gateway 320. The dialysis machine 302 may adjust the work flow on a GUI of the display to account for the additional medication step to remind the patient. Additionally, and/or alternatively, the mobile device 420 of the patient may receive the reminder via a mobile telecommunication network and display the reminder and/or transmit the reminder for display on the dialysis machine 302 when in proximity to the gateway 320 and/or the dialysis machine 302 via Wi-Fi, Bluetooth or other local area network.

### Example 3: Reminder of Effluent Sampling

The network messaging aspects of the system described herein may be used to transmit information on periodic effluent sampling that is to be performed by the patient and the required setup and dialysis machine parameters (e.g. collecting some fluid from initial drain, some from different drains etc.). In an embodiment, the dialysis machine 302 may receive the reminder for the effluent sampling that is transmitted remotely by the CIS 304 and requests the patient to do an effluent sample that night or some future night. In an embodiment, this could be an automatic process that is based on a calendar, and in some cases could be generated at the dialysis machine 302 and transmitted to the mobile device 420 of the patient as a reminder. The dialysis machine 302 may adjust the work flow on a GUI of the display to account for the effluent sampling to remind the patient. Additionally, and/or alternatively, the mobile device 420 of the patient may receive the reminder via a mobile telecommunication network and display the reminder and/or transmit the reminder for display on the dialysis machine 302 when in proximity to the gateway 320 and/or the dialysis machine 302 via Wi-Fi, Bluetooth or other local area network.

### Example 4: Special Instructions

The network messaging aspects of the system described herein may be used to transmit special instructions to a patient. For example, this could be reminders and or statements of encouragement for patients who have had trouble with certain steps. In an embodiment, the dialysis machine 302 may receive the special instructions via the gateway 320 that have been generated at the CIS 304 and transmitted via the CH system 300. The dialysis machine 302 may display the special instructions to remind the patient. For example, the display on the home dialysis machine 302 may be: “You haven't been draining well the past two nights, take 2 tablespoons of Lactulose in the morning.” The special instructions may also include general concern questions that may be answered via the GUI at the dialysis machine 302 by the patient, such as “How are you doing today?” In other embodiments, the special instructions may include additional treatment management steps, such as exit site assessment, may include photos or other pictures that inform the patient about the special instructions, and may enable the patient to make selections via the GUI (such as, “Which photo best corresponds to your exit site”) to continue through the special instructions. The system may thereby ask questions and capture patient answers. Additionally, and/or alternatively, the mobile device 420 of the patient may receive the reminder via a mobile telecommunication network and display the reminder and/or transmit the reminder for display on the dialysis machine 302 when in proximity to the gateway 320 and/or the dialysis machine 302 via Wi-Fi, Bluetooth or other local area network.

### Example 5: Patient Self Notes or Responses

The network messaging aspects of the system described herein may be used to allow patients to leave themselves notes or reminders and/or to enable the patient to respond back to a reminder message to give input.

In an embodiment, the patient may enter the notes via the dialysis machine 302. In various embodiment, the patient reminders may be displayed on the dialysis machine 302 at a later point in time in connection with a treatment, may be transmitted via directly or via the gateway 320 to the mobile device 420 of the patient, and/or may be transmitted via the CH system 300 to the CIS 304 or other external computer system. This could replace the need for physical “sticky notes” on a dialysis machine. In various embodiments, the notes may include audio or video reminders recorded by the patient at the dialysis machine 302 and/or may include handwritten notes written using a touch screen of the dialysis machine 302. Additionally, and/or alternatively, the mobile device 420 of the patient may receive the notes via a mobile telecommunication network and display the notes and/or transmit the reminder for display on the dialysis machine 302 when in proximity to the gateway 320 and/or the dialysis machine 302 via Wi-Fi, Bluetooth or other local area network.

In another embodiment, the patient can respond back to the reminder message to give an input, such as an acknowledgement of a reminder; a “Snooze” request to be reminder again in a set amount of time; and/or a data input response. For example, in response to a reminder message to a patient to perform a manual exchange, the patient could send a pre-set response and/or answer questions about the exchange, such as drain volume, time etc. Alternatively, this data capture may be done via an app on the mobile device of the patient which communicates with the system as further described in detail elsewhere herein. In an embodiment, the mobile phone of the patient may communicate the patient input to the dialysis machine when in proximity thereto and then the patient confirms the input responses on the dialysis machine.

Embodiments or implementations discussed herein may be combined with each other in appropriate combinations in connection with the system described herein. Additionally, in some instances, the order of steps in the flow diagrams, flowcharts and/or described flow processing may be modified, where appropriate. The system may further include a display and/or other computer components for providing a suitable interface with a user and/or with other computers.

Aspects of the system described herein may be implemented or controlled using software, hardware, a combination of software and hardware and/or other computer-implemented or computer-controlled modules or devices having described features and performing described functions. Data exchange and/or signal transmissions to, from and between components of the system may be performed using wired or wireless communication. This communication may include use of one or more transmitter or receiver components that securely exchange information via a network, such as the Internet, and may include use of components of local area networks (LANs) or other smaller scale networks, such as Wi-Fi, Bluetooth or other short range transmission protocols, and/or components of wide area networks (WANs) or other larger scale networks, such as mobile telecommunication networks.

Software implementations of aspects of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media, an SD card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system. The meanings of any method steps of the invention(s) described herein are intended to include any suitable method of causing one or more parties or entities to perform the steps unless a different meaning is expressly provided or otherwise clear from the context.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or equivalent thereof, should be generally construed to include [a] alone, [b] alone, [c] alone, or any combination of [a], [b] and [c].

Embodiments and implementations of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A medical system, comprising:
a medical device having a first display;
a processing device remotely located from the medical device; and
a network messaging system that transmits a message over a network between the medical device and the remote processing device, wherein the message includes information concerning a medical treatment performed by the medical device.

2. The medical system of claim 1, wherein the medical device is a dialysis machine and the medical treatment is a dialysis treatment.

3. The medical system of claim 1, wherein the medical device is disposed in a home of a patient.

4. The medical system of claim 1, further comprising:
a gateway device that facilitates a wireless network connection with the medical device.

5. The medical system of claim 4, wherein the gateway, the medical device and the processing device are joined to a same wireless local area network.

6. The medical system of claim 1, wherein the processing device is a mobile device.

7. The medical system of claim 1, wherein the processing device is a server.

8. The medical system of claim 1, wherein the message includes an instruction or a reminder to a patient concerning the medical treatment.

9. The medical system of claim 1, wherein the message includes one or more of an audio or video message.

10. The medical system of claim 1, wherein the message is interactive and requires at least one input from the patient.

11. The medical system of claim 1, wherein the message is provided over the Internet via a network infrastructure.

12. A network messaging system for a medical device, comprising:
a message-generating component that transmits a message over a network relating to a medical treatment performed by the medical device;
a message-receiving component that receives the message;
a gateway device that facilitates a wireless connection to the medical device, wherein the message is transmitted by the gateway device to or from the medical device.

13. The network messaging system of claim 12, wherein the medical device is a dialysis machine and the medical treatment is a dialysis treatment.

14. The network messaging system of claim 12, wherein the gateway and the medical device are disposed in a home of a patient.

15. The network messaging system of claim 12, wherein the message-generating component is a component of the medical device.

16. The network messaging system of claim 12, wherein the message-receiving component is a component of the medical device.

17. The network messaging system of claim 12, wherein the message-receiving component is a component of a mobile device.

18. The network messaging system of claim 12, wherein the message includes an instruction or a reminder to a patient concerning the medical treatment.

19. The network messaging system of claim 12, wherein the message includes one or more an audio message, a video message or an interactive message that requires at least one input from the patient.

20. The network messaging system of claim 12, wherein the message is provided over the Internet via a network infrastructure.
